(19) United States
(12) Patent Application Publication
(10) Pub. No.: US 2026/0308665 A1
(43) Pub. Date: Oct. 8, 2026

(54) METHODS OF SELECTING PRIMERS FOR AMPLIFICATION OF TARGET SEQUENCES

(71) Applicant: Ribbon Bio GmbH, Vienna (AT)

(72) Inventor: Gregory Linshiz, Vienna (AT)

(21) Appl. No.: 19/638,468

(22) Filed: Apr. 3, 2026

Related U.S. Application Data
(60) Provisional application No. 63/783,706, filed on Apr. 4, 2025.

(51) Int. Cl. C12Q 1/6811; G16B 30/00

(52) U.S. Cl. CPC C12Q 1/6811; G16B 30/00

(57) ABSTRACT

The invention provides methods and systems for designing and selecting primer pairs for targeted nucleic acid molecule amplification. The methods and systems of the invention enable detection and amplification of error-free molecules or molecules with specified genetic variations.

### BACKGROUND

Nucleic acid amplification techniques have been cornerstone methodologies in molecular biology since the development of Polymerase Chain Reaction (PCR) in 1983. While these techniques have revolutionized genetic research, diagnostic development, and personalized medicine, they continue to be hampered by fundamental limitations in primer design and sequence-specific amplification strategies.

The process of designing effective primer pairs has historically been a complex and technically challenging endeavor. Conventional primer design approaches have relied on relatively simplistic methods that fail to capture the intricate molecular dynamics of nucleic acid interactions.

Conventional primer design methods frequently result in off-target amplification, where primers bind to unintended genomic regions. This leads to reduced experimental accuracy, increased background noise, and compromised research and diagnostic reliability. Conventional methods also struggle to differentiate between error-containing and error-free molecular sequences. This prevents precise genetic variation detection, selective amplification of specific genetic variants, and accurate representation of molecular populations. These conventional methods further demonstrate significant weakness in detecting subtle genetic variations, designing primers that can discriminate between nearly identical sequences, and adapting to diverse genomic contexts and mutation landscapes.

The rapidly evolving landscape of genomic research, precision medicine, and molecular diagnostics necessitates a novel approach to primer design and selection that can address these fundamental challenges.

### SUMMARY

Methods and systems of the present disclosure enhance the efficiency of the isolation of error-free DNA molecules from a heterogeneous pool of sequences. By analyzing sequencing data, optimal barcode pairs, also potentially serving as PCR primer pairs, are identified that specifically amplify molecules matching a target reference sequence with high fidelity. Methods and systems of the present disclosure employ algorithms to score and rank barcode pairs based on factors like abundance, specificity, and reads sequence similarity to the target reference sequence. Through this selection process, methods and systems of the present disclosure enable the selective amplification of error-free DNA molecules, thereby improving the accuracy and reliability of downstream applications, e.g., nucleic acid assembly. The methods and systems of the present disclosure provide targeted primer pairs that facilitate the selective amplification of desired sequences, ensuring that only error-free molecules or molecules with any desired genetic variant (e.g., Single-nucleotide polymorphisms (SNPs)) are amplified. The invention provides cell-free molecular cloning approaches based on molecular mining PCR (mmPCR) to identify and amplify error-free DNA molecules from a diverse sequence pool, thereby eliminating the need for bacterial clonal selection. In a preferred instance, DNA fragments are first tagged with uniquely-designed, orthogonal barcode sequences that serve as both unique molecular identifiers and precise binding sites for predesigned primers. The concentration of these tagged molecules is accurately measured and then diluted to achieve an optimal range of several thousand molecules per reaction, ensuring consistent and robust amplification. Universal outer primers are used to amplify this diluted, tagged pool, and the resulting product is subjected to high-throughput sequencing. Advanced computational methods analyze the sequencing data to generate mapping scores that quantify how closely each amplified molecule matches a target reference sequence. Barcode pairs associated with molecules displaying the highest mapping scores are then selected as specific amplification primers to enrich only those molecules that meet a stringent accuracy threshold, achieving up to tenth thousand fold target enrichment.

The optimal barcode pairs as taught in the present invention, also serving as binding sites for PCR primers may be short DNA sequences designed to specifically target a specific region of DNA for amplification in a PCR reaction, while also containing unique barcode sequences that allows for identification of individual DNA molecules analyzed in a heterogenous pool of DNA molecules. The barcode functions as both a target-specific amplification tool (e.g., by serving as a primer binding site) and a label for molecule identification (e.g., contain unique sequences allowing for identification of individual molecules in a heterogenous pool).

In certain aspects, the invention provides methods and systems for primer selection. Preferred methods include selecting primer pairs from a database; making copies, using the selected primer pairs, from among a heterogeneous pool of DNA molecules; sequencing the copies to produce sequence data; mapping the sequence data to a target reference sequence, thereby generating mapping scores; and identifying, based on the mapping scores, a primer pair for amplifying the target reference sequence.

In certain embodiments, a nested approach may be employed. This method utilizes universal outer primer pairs to general multiple clonal copies of the target sequence. The universal outer primers are designed to anneal to conserved regions flanking the target sequence, ensuring consistent amplification across various samples. Once the initial amplification is complete, selected barcode (BC) primer pairs are then used to target and selectively amplify the desired clonal copy from the pool of generated sequences. These BC primer pairs are unique to each specific clonal copy and allow for the identification and isolation of the target sequences.

Primers are short, single-stranded nucleic acid sequences, typically 18-30 nucleotides in length, that serve as an initial binding site for DNA polymerase during nucleic acid amplification. Primers provide a 3′ hydroxyl group from which DNA synthesis can commence, enabling targeted replication of specific genetic sequences.

Copies refer to the replicated nucleic acid molecules generated through an amplification process, e.g., those produced by enzymatic replication mechanisms such as Polymerase Chain Reaction (PCR). Each copy represents a duplication of the original template sequencing, potentially maintaining or introducing specific genetic characteristics.

A heterogenous pool of DNA molecules is a complex mixture of nucleic acid sequences that may be characterized by genetic variation between individual molecules, potential presence of error-containing sequences, diverse molecular origins, varying levels of integrity and completeness, and/or multiple sequence variants within a single sample.

Sequencing is a molecular biology technique for determining the precise nucleotide arrangement within a DNA or RNA molecule. Sequencing may involve the identification and documentation of the linear sequence of nucleotides (adenine, thymine, guanine, cytosine) that comprise a genetic molecule. In addition to the standard nucleotides, sequencing technologies that are capable of capturing modified bases can also be used to identify variations such as deaminated or methylated bases including methylated cytosine (5-methylcytosine or 5mC), hydroxymethylated cytosine (5-hydroxymethylcytosine or 5hmC), and methylated adenine (6-methyladenine or 6 mA).

Sequence data represents the digital or molecular representation of nucleotide arrangements, including raw nucleotide sequence information, associated quality metrics, genomic context, potential genetic variations, and metadata describing molecular characteristics.

A mapping score may represent a multidimensional evaluation of sequence similarity, genetic variation magnitude, molecular reproduction accuracy, and or potential amplification artifacts.

Amplifying is the process of enzymatically generating multiple copies of a specific nucleic acid sequence through iterative replication mechanisms. This process may involve precise molecular replication, exponential generation of genetic material, selective targeting of specific genomic regions, and/or controlled enzymatic extension of primer-initiated sequences.

The selecting step may involve screening of a database containing known forward and reverse barcode sequences (e.g., orthogonal forward and reverse barcode sequences). The selected primers may be subsequently deployed to generate molecular copies of a desired target DNA molecule within a heterogenous DNA molecule pool via an amplification process. The amplification process utilizes enzymatic replication mechanisms, e.g., utilizing DNA polymerase to generate molecular copies. The amplification process may involve a multiplexing approach comprising the selection and utilization of multiple primer pairs. The amplified molecular copies may undergo high-throughput sequencing, generating sequence data. The generated sequence data may be computationally mapped against a target reference sequence, creating a mapping score that serves as an evaluation metric. The mapping process may involve algorithmic alignment of generated sequences, statistical probability calculations of sequence matches, quantitative assessment of genetic variation, and/or computation prediction of molecular fidelity. Based on the generated mapping scores, an advanced selection algorithm may identify an optimal primer pair. This selection algorithm may involve integration of multiple scoring metrics, prediction of amplification efficiency, and/or minimization of potential experimental variability.

In certain embodiments, the selecting step comprises using an Aho-Corasick automaton. The Aho-Corasick automaton is an algorithmic approach for multiple pattern matching within complex genetic sequences. It enables simultaneous matching of multiple primer sequences, provides rapid, concurrent sequence identification, minimizes computational complexity in large-scale genetic datasets, and allows for efficient pattern recognition across heterogenous molecular populations.

In certain embodiments, the selecting step comprises using k-mer based indexing combined with Hamming distance verification. This involves fragmenting genetic sequences into fixed-length subsequences (k-mers), creating a comprehensive molecular index, implementing Hamming distance calculation to assess genetic variations, and quantifying genetic sequence similarities. Hamming distance verification allows for measuring genetic sequence divergence, identifying molecular variations, quantifying genetic sequence similarities, and providing a computational framework for sequence comparison.

In certain embodiments, the selecting step comprises Levenshtein distance calculation. The Levenshtein distance, is a measure of similarity between two strings, quantified as the minimum number of single-character edits (e.g., insertions, deletions, or substitutions) required to change one string into the other. In the present disclosure, this calculation plays a role in identifying primers that closely match the target reference sequence while minimizing mismatches. The Levenshtein distance may be used to evaluate the similarity between the selected primers and the target reference sequence. For example, selecting primers with the lowest Levenshtein distance, may result in the maximum likelihood of successful hybridization and amplification.

In certain embodiments, the mapping step further comprises detecting variants. In certain embodiments, the variants are selected from the group comprising substitutions, insertions, and deletions. Types of genetic variations include substitutions, insertions, deletions, and modified or damaged bases (e.g., deaminated or methylated bases). Damaged bases may include, for example, deaminated cytosine (uracil). Substitutions involve single nucleotide replacements, insertions involve the addition of genetic material, and deletions involve the removal of genetic sequences. Complex variations involve combinations of multiple mutation types.

In certain embodiments, the mapping scores are generated based on, in part, types and frequencies of the variants detected during the mapping step. The analysis of variant frequencies may involve statistical modeling of variant distribution, computational prediction of variant emergence probabilities, and quantitative assessment of genetic variation magnitudes.

In certain embodiments, the primer pair for amplifying the target reference sequence has a mapping score exceeding a minimum threshold. In certain embodiments, the identifying step comprises excluding sequences with high-frequency variants.

A mapping score threshold system may be developed that establishes minimum acceptable mapping scores, implements adaptive scoring mechanisms, filters out low-quality molecular sequences, and provides quantitative quality control. The mapping score threshold system may exclude sequences with high-frequency variants, undesired statistically significant molecular aberrations, and potential amplification artifacts.

In certain embodiments, each of the DNA molecules are labeled with barcodes. In certain embodiments, the barcodes are orthogonal barcodes. Utilizing orthogonal barcodes provide unique molecular identification, enhanced multiplexing capabilities, molecular tracking, and validation mechanisms. Orthogonal barcodes (which are not susceptible to sequencing errors) enable the simultaneous analysis of multiple molecular populations, computational differentiation of molecular sources, enhanced experimental reproducibility, and advanced molecular population management.

### DETAILED DESCRIPTION

The present disclosure provides methods of isolating error-free DNA molecules or molecules with desired sequence variants (e.g., single nucleotide polymorphisms (SNPs)) from a heterogeneous pool of sequences. By analyzing sequencing data, methods of the present invention identify optimal barcode pairs, which correspond to PCR primer pairs, that specifically amplify molecules matching a reference sequence with high fidelity. Methods of the present disclosure use algorithms to score and rank barcode pairs based on factors such as abundance, specificity, and sequence similarity to the reference sequence. Methods of the present disclosure allow for the selective amplification of error-free DNA molecules or those containing specific sequence variants (e.g., SNPs), thereby improving the accuracy and reliability of downstream applications, e.g., nucleic acid assembly. Thus, methods of the present disclosure provide targeted primer pairs that facilitate the selective amplification of desired sequences, ensuring that error-free molecules or molecules with specified genetic variations are amplified.

Methods and systems of the present disclosure involve a heterogeneous pool of DNA molecules. Each DNA molecule may be labeled or tagged with predesigned orthogonal forward and reverse barcodes. A pool of DNA molecules containing the forward and reverse barcodes, may be generated via nucleic acid amplification techniques such as, Multiplex PCR, Real-Time PCR, Reverse Transcription PCR, Nested PCR, Loop-Mediated Isothermal Amplification, Helicase-Dependent Amplification, Nucleic Acid Sequence-Based Amplification (NASBA), Multiple Displacement Amplification (MDA), and Ligase Chain Reaction. Orthogonal primer pairs are designed to only hybridize with their corresponding barcode sequences and not with any other barcode sequences, and minimize the potential for cross-hybridization with other oligonucleotide sequences. This selective binding ensures that each primer pair only amplifies its intended target, thereby preventing mispriming. These barcodes uniquely tag each molecule, facilitating identification and tracking throughout the method and system. Additionally, a target reference sequence may be provided, representing the target DNA sequence that the molecules from the pool are expected to match or closely resemble.

Methods and systems of the present disclosure may involve a method for generating orthogonal primers. The method may comprise generating a plurality of primer pairs specific to different target sequences, wherein each primer pair comprises a forward and a reverse primer that are complementary to the target sequence and do not have significant homology with each other.

In certain embodiments, orthogonal barcodes serve as the binding sites for PCR primers during dial-out PCR. Dial-out PCR is a method for retrieving accurate DNA molecules for gene synthesis. A complex library of DNA molecules is modified with unique flanking tags before massively parallel sequencing. Tag-directed primers then enable the retrieval of molecules with desired sequences by PCR. Dial-out PCR enables multiplex in vitro clone screening.

The optimal barcode pairs of the present invention, also serving as PCR primer pairs, may be short DNA sequences designed to specifically target a specific region of DNA for amplification in a PCR reaction, while also containing unique barcode sequences that allows for identification of individual DNA molecules analyzed in a heterogenous pool of DNA molecules. The barcode functions as both a target-specific amplification tool and a label for molecule identification. The barcode pairs of the present invention, may also serve as binding sites for PCR primer pairs. The PCR primer pairs may be orthogonal PCR primer pairs.

The methods and systems of the present disclosure further comprise identifying, pairing, and/or selecting forward and reverse barcodes within sequencing reads. By selecting these barcode pairs, the system can accurately determine the boundaries of each individual DNA molecule within the complex mixture. This step is for isolating the sequences of interest, e.g., target reference sequence, from a vast array of sequencing data.

The method further comprises utilizing the positions of the matched barcodes to isolate the target reference sequences corresponding to each barcode pair. This extraction ensures that only the relevant portions of the sequencing reads, i.e., those between the forward and reverse barcodes, are considered for further analysis.

The extracted sequences are aligned to the provided reference sequence using global alignment algorithms. This alignment enables the detection of any sequence variations, such as substitutions, insertions, or deletions, by comparing each extracted sequence to the reference at a nucleotide level.

Following alignment, the methods and systems of the present disclosure identify all observed variants across the dataset. The methods and systems of the present disclosure comprise cataloguing substitutions, insertions, and deletions and compiling their occurrences and frequencies. This aggregation of variant data plays a role in understanding the diversity within the DNA pool and for assessing the fidelity of each molecule relative to the reference sequence.

The methods and systems of the present disclosure further comprise quantifying the overall similarity of each extracted sequence to the target reference sequence. The methods and systems of the present disclosure consider the types and frequencies of detected variants, assigning penalties or weights based on the nature of each variant. The resulting similarity scores provide a quantitative measure of how closely each molecule matches the reference, with higher scores indicating greater similarity and, therefore, higher desirability for amplification.

The methods and systems of the present disclosure further comprise utilizing a mapping score, which includes the similarity scores and other metrics, to choose the optimal barcode pairs, which correspond to PCR primer pairs. By selecting barcode pairs associated with sequences that closely match the reference, the methods and systems of the present disclosure ensure that the primers will specifically amplify error-free molecules or those containing desired sequence variants (e.g., single nucleotide polymorphisms (SNPs)). This targeted selection enables the amplification of specific DNA molecules from the heterogeneous pool.

The methods and systems of the present disclosure are capable of handling large sequencing datasets of millions of reads efficiently through parallel processing and optimized data structures.

### Barcode Selection

Methods and systems of the present disclosure enable the identification of target reference sequences based on known barcode patterns. Methods and systems of the present disclosure may, in some embodiments, comprise creating a database containing forward and reverse barcode sequences. These sequences serve as references for identifying barcodes within the sequencing reads. In certain aspects, methods involve randomly attaching barcodes to DNA molecules in a pool, detecting forward and reverse barcodes in all reads, clustering reads based on barcode similarities, scoring barcode pairs and selecting potential clusters, mapping the read sequences from the top clusters to reference (target) sequences, scoring the variants by comparing reads to the reference sequences, and selecting the optimal barcode pair (primers) to amplify the desired error-free molecutls from the pool base on overall scores and thresholds.

To detect barcodes, the method and/or system may employ a three-step strategy. In the first step, the system and/or method may use the Aho-Corasick algorithm. The Aho-Corasick automaton is a string-matching algorithm capable of finding all occurrences of multiple patterns in a text simultaneously. It constructs a finite automaton from the set of barcode sequences and scans the read in a single pass, checking for any matches against this automaton. An advantage of using the Aho-Corasick algorithm lies in its linear time complexity relative to the size of the input text and the number of patterns. Its time complexity is (O(n+m+z)), where (n) is the length of the text (the sequencing read), (m) is the total length of all the barcode patterns, and (z) is the number of matches found. By utilizing the Aho-Corasick algorithm, the methods and systems of the present disclosure can identify barcodes that align with the known sequences.

In certain embodiments, the system proceeds to the second step, which employs k-mer based matching combined with Hamming distance verification. A k-mer refers to a subsequence of length (k) extracted from the barcode sequences. The value of (k) is chosen based on the typical length of the barcodes and the expected error rate in the sequencing data. The methods and systems of the present disclosure construct an index of all possible k-mers derived from the known barcodes. It then scans the sequencing read to identify any k-mers that match those in the index, narrowing down potential regions where a barcode may be located. Once potential barcode regions are identified through k-mer matching, the methods and systems of the present disclosure verify these candidates by calculating the Hamming distance between the candidate sequence and the corresponding barcode sequence. The Hamming distance is defined as the number of positions at which the corresponding symbols are different between two sequences of equal length. The methods and systems of the present disclosure accept a barcode match if the Hamming distance is below a predefined threshold, indicating that the candidate sequence is sufficiently similar to the known barcode despite minor discrepancies. The process of generating k-mers and indexing them has an initial overhead proportional to the total number of k-mers, which is (O(m)), where (m) is the total length of the barcode sequences. Scanning the read for k-mer matches operates in (O(n)) time, where (n) is the length of the read, since each position in the read is considered once. Calculating the Hamming distance between sequences of length (l) is (O(l)). Given that barcodes are relatively short (typically around 20 bases), this computation is fast and may not impact overall performance. In the k-mer based matching step, after identifying potential barcode regions through k-mer matches, the methods and systems of the present disclosure verify these candidates using the Hamming distance. Since the Hamming distance is only defined for sequences of equal length and only accounts for substitutions, it provides a quick and efficient method for assessing similarity when insertions and deletions are not expected or are negligible. By setting a threshold for the maximum allowable Hamming distance, the methods and systems of the present disclosure can filter out candidates that are unlikely to be true barcode matches, reducing false positives.

In certain embodiments, methods and systems of the present disclosure proceed to the third step, which involves Levenshtein distance (edit distance) matching. The Levenshtein distance extends beyond substitutions to include insertions and deletions. In this step, the methods and systems of the present disclosure compare segments of the sequencing read to each barcode sequence using algorithms that compute the Levenshtein distance. A dynamic programming approach may be used to fill a matrix where each cell represents the minimum edit distance between prefixes of the two sequences up to that point. The methods and systems of the present disclosure accept a barcode match if the Levenshtein distance between the read segment and the barcode is within an acceptable range, indicating that the barcode is present despite potential insertions, deletions, or substitutions caused by sequencing errors or mutations. The classic dynamic programming algorithm for computing the Levenshtein distance between two sequences of lengths (n) and (m) has a time complexity of (O(nm)). However, optimizations are possible when the maximum allowable edit distance is small (e.g., due to short barcodes and low error tolerance). Techniques such as the Levenshtein automaton or banded algorithms may reduce the average computation time. As the methods and systems of the present disclosure need to compare the barcode sequences against multiple possible positions within the read, the computational load increases proportionally to the length of the read and the number of barcodes.

The three-step matching strategy is designed to optimize both computational efficiency and matching sensitivity. By first attempting exact matching using the Aho-Corasick algorithm, the methods and systems of the present disclosure capitalize on the speed of this method to quickly process the bulk of the data where barcodes are intact and unaltered. When exact matching fails, e.g., due to minor sequencing errors causing substitutions, the k-mer based matching with Hamming distance verification provides a balance between speed and error tolerance. It narrows down candidate regions and verifies them with minimal computational overhead. Finally, the Levenshtein distance matching serves as a comprehensive method for detecting barcodes that have undergone alterations, such as insertions or deletions. While more computationally intensive, it ensures that valuable data is not lost due to sequencing artifacts. By structuring the barcode detection process in this way, the methods and systems of the present disclosure prioritize computational resources, applying more intensive methods only when necessary. This approach ensures that the barcode detection is both accurate and efficient, capable of handling large datasets typical in high-throughput sequencing applications. The combined use of these three methods allows the methods and systems of the present disclosure to maintain high throughput without compromising on the quality of barcode detection. Aho-Corasick Exact Matching handles the majority of cases with linear time complexity and minimal overhead. K-mer Matching with Hamming Distance Verification introduces a moderate computational load, proportional to the number of k-mers and the read length, but remains efficient due to the simplicity of Hamming distance calculations. Levenshtein Distance Matching may be applied selectively when previous methods fail. Its higher computational cost is justified by the need for greater sensitivity in detecting complex barcode variations. By optimizing each step and employing them sequentially, the methods and systems of the present disclosure ensure that barcode detection is both robust and scalable, suitable for processing the vast amounts of data generated in modern sequencing projects.

### Sequence Data

Once barcodes are matched, the system proceeds to extract sequence data located between the paired forward and reverse barcodes. The positions of the barcodes define the boundaries of the target sequence within each read. The system applies length filtering to ensure that only sequences within a specified range are considered. The extraction algorithm accounts for cases where barcodes may overlap or be located in unexpected positions due to insertions, deletions, or other anomalies in the sequence data.

### Sequence Alignment

The extracted sequences are then aligned to a target reference sequence using global pairwise alignment algorithms. The Needleman-Wunsch algorithm is employed for this purpose, providing alignments by considering the entire length of the sequences. The scoring scheme used in the alignment process includes rewards for matches and penalties for mismatches and gaps, with specific values assigned: a match score of +2, a mismatch penalty of −1, a gap opening penalty of −0.5, and a gap extension penalty of −0.1. To enhance performance, the alignment tasks are parallelized by distributing the workload across multiple processors.

### Variant Detection and Aggregation

After alignment, the methods and systems of the present disclosure compare the aligned sequences to the reference to identify sequence variants, including substitutions, insertions, and deletions. Each variant is recorded with detailed information such as its type, position, length, and the specific sequence involved.

Variants are aggregated across all sequences to determine their frequency and distribution. This aggregation plays a role in assessing the overall impact of variants on the target sequences and for informing the similarity score calculation. Efficient data structures, such as nested dictionaries, are used to store and retrieve variant data quickly.

The methods and systems of the present disclosure are designed to handle complex variants, including multiple consecutive insertions or deletions and overlapping variants. It ensures accurate counting and characterization of variants even in the presence of sequencing errors.

### Similarity Score Calculation

The similarity score quantifies how closely the extracted sequences match the reference, accounting for the types and frequencies of observed variants. The score calculation involves assigning penalties to each variant based on predefined weights and adjusting these penalties according to the complexity and length of the variant sequences.

The similarity score is calculated using the formula:

$$Similarity Score = max ( 0 , 100 - ( \frac{Capped Total Penalty}{Max Total Penalty} \times 100 ) )$$

The total penalty is the sum of penalties for all variants:

$$Total Penalty = \sum ( Wtype \times Cvariant \times {L}_{variant} )$$

where (Wtype) is the weight assigned to the variant type (substitution, insertion, deletion), (Cvariant) is the complexity of the variant sequence, and (Lᵥₐᵣᵢₐₙₜ) is the length of the variant. The total penalty is capped at 50% of the maximum possible penalty to prevent excessive impact on the similarity score.

Variant weights are assigned as follows: substitutions carry a weight of 15 units per occurrence, insertions have a weight of 10 units per complexity-adjusted length, and deletions also have a weight of 15 units per complexity-adjusted length. The complexity of insertion and deletion sequences is measured using Shannon entropy, which assesses the diversity of nucleotide composition within the sequence. A higher entropy value indicates a more complex sequence with greater nucleotide diversity.

The entropy (H) is calculated using the formula:

$$H = - {\sum }_{i = 1}^{n} {p}_{i} {log}_{2} {p}_{i}$$

where (pᵢ) is the probability of nucleotide (i) in the sequence, and (n) is the number of unique nucleotides present. The entropy is normalized by dividing by the maximum possible entropy for the sequence length. Complex sequences are penalized less in the similarity score calculation, as they are less likely to result from sequencing errors or homopolymer runs.

### Scoring and Selection of Barcode Pairs

The methods and systems of the present disclosure may employ a multi-faceted scoring algorithm that integrates various metrics to assess the quality and suitability of barcode pairs.

To quantitatively evaluate each barcode pair, the methods and systems of the present disclosure may calculate a Selection Score, also referred to as a Mapping Score, that combines multiple factors reflecting the pair's abundance, uniqueness, and read quality. The calculation is performed using the following formula:

$$Selection Score = - {w}_{5} \times ( Frw Sharing Count + Rev Sharing Count ) + {w}_{a} \times Abundance Percentage + {w}_{r} \times Reads Ratio + {w}_{sr} \times Reads Sharing Ratio$$

Where wₛ is the sharing pairs weight, penalizing barcode pairs that share barcodes with multiple other pairs, which could indicate non-specific binding; Frw Sharing Count and Rev Sharing Count represent the number of times the forward and reverse barcodes are shared with other pairs, respectively; wₐ is the abundance weight, rewarding pairs that are abundantly represented in the dataset; Abundance Percentage is the percentage of reads associated with the barcode pair that fall within the desired target length range; wᵣ is the ratio weight, emphasizing the proportion of reads within the target length compared to all reads for the pair; Reads Ratio is the ratio of reads within the target length range to the total reads for the pair; wₛᵣ is the sharing reads weight, considering the uniqueness of reads associated with the barcode pair; and Reads Sharing Ratio measures the proportion of reads unique to the barcode pair versus those shared with other pairs.

The selection score, or mapping score, is rounded and converted to an integer for consistency. By incorporating both positive and negative factors, the score balances the desire for abundant, unique barcode pairs with the need to avoid pairs that might contribute to non-specific amplification.

After calculating the selection scores, the methods and systems of the present disclosure may filter out barcode pairs that do not meet a minimum threshold for the number of reads within the target length range. This step ensures that only pairs with sufficient data to support reliable analysis are considered. The filtered dataset is then sorted in descending order based on the selection scores. The top n barcode pairs are selected as the best candidates.

To further refine the selection, the methods and systems of the present disclosure may perform variant calling on the sequences associated with the best barcode pairs. This process involves aligning the sequences to a reference and identifying any sequence variants, such as substitutions, insertions, or deletions.

For each barcode pair, a Similarity Score may be calculated based on the extent to which the associated sequences match the reference sequence. The score considers the number and types of variants detected, penalizing pairs with higher numbers of mismatches or errors. A higher similarity score indicates that the sequences amplified by the barcode pair closely match the reference, which is desirable for specific and accurate amplification.

The similarity scores may be integrated into the selection scores, or mapping scores, to enhance the overall evaluation of each barcode pair. By assigning a weight to the similarity score wₛ. The methods and systems of the present disclosure adjust the selection scores to favor barcode pairs that not only have favorable abundance and uniqueness metrics but also produce sequences that closely match the reference.

The updated selection score is calculated by adding the weighted similarity score to the existing selection score for each barcode pair:

$$Updated Selection Score = Selection Score + {w}_{s} \times Similarity Score$$

Where wₛ is the weight assigned to the similarity score.

With the similarity scores integrated, the methods and systems of the present disclosure may apply additional filtering criteria to identify the best barcode pairs for automation, e.g., (a) a Similarity Score Threshold: Barcode pairs must have a similarity score exceeding a minimum threshold, and/or (b) a Maximum Variant Frequency: Pairs are filtered based on the maximum frequency of any variant detected, ensuring that sequences with high-frequency variants, which may indicate systematic errors or polymorphisms, are excluded.

The filtered barcode pairs constitute the final candidates for amplification. If the number of candidates is less than the desired number, the methods and systems of the present disclosure may report this, indicating that fewer pairs met the stringent criteria.

The multi-parameter scoring system allows for prioritization of primer pairs with a higher abundance to ensure that the selected primers will be effective across a significant portion of the dataset (e.g. molecules), penalization of shared barcodes and reward for unique reads reduces the risk of non-specific amplification, which can lead to low sequence purity of the amplicons, and integration of similarity scores to ensure that the sequences amplified by the selected barcode pairs closely match the reference, minimizing errors and increasing the reliability fishing out desired sequences.

By combining these factors into a single mapping score, or single selection score, the methods and systems of the present disclosure rank barcode pairs, enabling the identification of the suitable candidates for primer pair selection.

### Examples

### 1. Tiered Assembly

One downstream application that may benefit from the methods and systems of the present disclosure may involve nucleic acid assembly. A computer system may use associated liquid handling hardware to make a mixture comprising a first oligonucleotide, a second oligonucleotide, and a ligase and then ligate the first oligonucleotide to the second oligonucleotide to make a first intermediate construct. Where the first and second oligonucleotides are both e.g., N-mer constituent oligos, that first set of mixing and ligating operations may be described as a first ‘tier’ of assembly. The computer system may then direct a second tier of assembly by ligating the first intermediate construct to a second intermediate construct that includes a third oligonucleotide ligated to a fourth oligonucleotide to provide a first third-tier product. The steps may be nested, and the computer system may proceed by ligating the first third-tier product to a second third-tier product to yield a fourth-tier product; and ligating the fourth tier product to an assemblage of the oligonucleotides to yield the contiguous nucleic acid molecule, aka the DNA product.

After ligating the N-tier products together to create the DNA product, the method may include other steps such as, for example, packaging the DNA product and storing it. The method may include copying or amplifying the DNA product (e.g., by PCR or rolling circle amplification). The method may include synthesizing a large number of copies of the DNA product simultaneously (e.g., by performing the mixing and ligation steps on volumes of the constituent oligos). Whether by making many synthetic DNA molecules simultaneously, or by making one and amplifying it, or a combination of those two, the method may include providing a large number (e.g., hundreds of thousands to millions) of clonal copies of the one DNA product, e.g., together in one container. The DNA product may be stored for a period, e.g., in ethanol, or frozen, or be lyophilized and stored dry. The DNA product may be subject to DNA sequencing e.g., via a sequencing instrument to confirm accuracy and purity of the product.

The methods and systems of isolating error-free DNA molecules or molecules with desired specified genetic variations from a heterogeneous pool of sequences of the disclosure may be used at any step of the nucleic acid assembly workflow. Methods of the present disclosure allow for the selective amplification of error-free DNA molecules or those containing specific sequence variants (e.g., SNPs), thereby improving the accuracy and reliability of downstream applications e.g. tiered nucleic acid assembly. Thus, methods of the present disclosure may be used at any step of the nucleic acid assembly workflow to provide targeted primer pairs that facilitate the selective amplification of desired sequences.

### 2. Polymerization-Based Assembly

Another downstream application that may benefit from the methods and systems of the present disclosure involves nucleic acid assembly through polymerization-based techniques. A computer system may use associated liquid-handling hardware to prepare a mixture containing a template strand of DNA, nucleotides, and a DNA polymerase enzyme. This system initiates the assembly by facilitating the binding of the primer to the template strand, which serves as a starting point for the polymerase enzyme. The polymerase then catalyzes the addition of complementary nucleotides to the growing strand, effectively synthesizing a new DNA molecule.

The computer system may control the temperature and cycling conditions, such as those used in PCR to optimize the extension process. This method allows for the construction of long DNA sequences from shorter template strands. The resulting synthesized DNA molecule can then be subject to further processing steps such as purification, sequencing, and storage.

The methods and systems of isolating error-free DNA molecules or molecules with desired specified genetic variations from a heterogeneous pool of sequences of the disclosure may be used at any step of the nucleic acid assembly workflow. Methods of the present disclosure allow for the selective amplification of error-free DNA molecules or those containing specific sequence variants (e.g., SNPs), thereby improving the accuracy and reliability of downstream applications e.g. polymerization-based nucleic acid assembly. Thus, methods of the present disclosure may be used at any step of the nucleic acid assembly workflow to provide targeted primer pairs that facilitate the selective amplification of desired sequences.

The methods and systems described in the present disclosure are designed to be versatile and applicable to various sources of DNA, encompassing both cell-free and cell-based processes. The described techniques can be utilized with DNA fragment pools derived from in vitro assembly, as well as with plasmid pools extracted from cell lysates. This flexibility ensures that the methods can be adapted to a wide range of experimental setups and workflows.

For cell-free applications, DNA fragments generated through in vitro synthesis or assembly can be directly incorporated into the described processes. These fragments can originate from diverse sources, including oligonucleotide pools, synthetic gene constructs, or assembled genetic elements.

In cell-based processes, plasmid pools obtained from cell lysates serve as a source of DNA. These plasmid pools can be prepared form various cell types, such as bacterial or eukaryotic cells, allowing for the amplification and assembly of genetic sequences present within cellular environments.

By accommodating both cell-free and cell-based sources of DNA, the methods and systems of the present disclosure provide a robust and adaptable framework for genetic analysis and manipulation. This versatility enhances the applicability of the techniques across different research and industrial contexts, ensuring their utility in a broad spectrum of genetic studies and biotechnological applications.

What is claimed is:

1. A method of primer selection, the method comprising:
selecting primer pairs from a database;
making copies, using the selected primer pairs, from among a heterogeneous pool of DNA molecules;
sequencing the copies to produce sequence data;
mapping the sequence data to a target reference sequence, thereby generating mapping scores; and
identifying, based on the mapping scores, a primer pair for amplifying the target reference sequence.

2. The method of claim 1, wherein the selecting step comprises using Aho-Corasick automaton.

3. The method of claim 1, wherein the selecting step comprises using k-mer based indexing combined with Hamming distance verification.

4. The method of claim 1, wherein the selecting step comprises Levenshtein distance calculation.

5. The method of claim 1, wherein the mapping step further comprises detecting variants.

6. The method of claim 5, wherein the variants are selected from the group comprising substitutions, insertions, and deletions.

7. The method of claim 5, wherein the mapping scores are generated based on, in part, types and frequencies of the variants detected during the mapping step.

8. The method of claim 1, wherein the primer pair for amplifying the target reference sequence has a mapping score exceeding a minimum threshold.

9. The method of claim 7, wherein the identifying step comprises excluding sequences with high-frequency variants.

10. The method of claim 1, wherein each of the DNA molecules are labeled with barcodes.

11. The method of claim 10, wherein the barcodes are orthogonal barcodes.
